(19) United States
(12) Patent Application Publication
(10) Pub. No.: US 2026/0198848 A1
(43) Pub. Date: Jul. 16, 2026

(54) REPLICATED DATA ARCHITECTURE FOR MULTIPLE IMPLANT APPLICATION

(71) Applicant: Warsaw Orthopedic, Inc., Warsaw, IN (US)

(72) Inventors: Elias Mulugeta Ayana, Blaine, MN (US); Newton H. Metcalf, JR., Memphis, TN (US); Adam D. Glaser, Collierville, TN (US); Jerald L. Redmond, Germantown, TN (US)

(21) Appl. No.: 19/267,704

(22) Filed: Jul. 14, 2025

Related U.S. Application Data
(60) Provisional application No. 63/745,012, filed on Jan. 14, 2025.

(51) Int. Cl. A61B 5/00; A61B 5/01; A61B 5/11; A61B 17/70; A61B 17/00; A61B 90/00

(52) U.S. Cl. CPC A61B 5/4848; A61B 5/01; A61B 5/1118; A61B 5/4561; A61B 17/7032; A61B 2017/00075; A61B 2017/00084; A61B 2017/00221; A61B 2017/00734; A61B 2090/061; A61B 2090/064; A61B 2562/0219

(57) ABSTRACT

Spinal implants and systems of spinal implants are disclosed. A spinal implant includes an attachment portion configured to attach to a spinal construct or a bone of a subject, a battery, at least one sensor configured to measure strain, temperature, position and/or acceleration of the implant, a transmitter electrically interfaced with an antenna and a processor. The processor is configured to receive, from at least one sensor, raw strain, position and/or acceleration information. The processor is further configured to execute one or more predefined algorithms, each predefined algorithm configured to process the raw information to determine a health parameter associated with the subject. The processor is further configured to cause the transmitter to transmit at least one determined health parameter to a remote device.

### CROSS-REFERENCE TO RELATED APPLICATION

This application claims the benefit of and priority to U.S. Provisional Patent Application No. 63/745,012 filed January 14, 2025, the entire disclosure of which is incorporated by reference herein.

### BACKGROUND

The present disclosure generally relates to mechanical and electrical sensor assemblies for implant devices, and more particularly to implants that transmit sensor data to an external reader device.

Treatment of spinal disorders, such as degenerative disc disease, disc herniations, scoliosis or other curvature abnormalities, fractures, and so forth often require surgical treatments. For example, spinal fusion may be used to limit motion between vertebral members. As another example, implants may be used to preserve motion between vertebral members.

Surgical treatment may involve the use of longitudinal members, such as spinal rods. Longitudinal members may be attached to the exterior of two or more vertebral members to assist with the treatment of a spinal disorder. Longitudinal members may provide a stable, rigid column that helps bones to fuse, and may redirect forces over a wider area away from a damaged or defective region. Also, rigid longitudinal members may help in spinal alignment.

Screw assemblies may be used to connect a longitudinal member to a vertebral member. A screw assembly may include a pedicle screw, hook, or other connector, among other components. Pedicle screws can be implanted, e.g., above and/or below vertebral members to be fused, and a longitudinal member can connect the pedicle screws to inhibit or control movement. A set screw can secure the longitudinal member to pedicle screws, hooks or other connectors.

Some implants include sensors configured to measure forces between components, temperature or other indication of infection at a single surgical site, and/or indications of post-operative progress. Reliably transmitting such measurements to an external device may be difficult. This may be particularly true for deeply implanted devices and/or space-constrained implants, which may be unable to transmit measurements to the external device due to signal attenuation and/or power limitations. Even those more shallow devices may have only sporadic connections to any external device. Therefore, there is a need to reliably manage measurements from “smart” implants until the measurements can be transmitted to the external device.

This document describes methods and systems that are directed to addressing the problems described above, and/or other issues.

### SUMMARY

The techniques of this disclosure generally relate to spinal implants and systems of spinal implants. Issues associated with prior solutions are addressed by the subject matter of the independent claims included in this document. Additional advantageous aspects are included in the dependent claims.

In one aspect, the present disclosure provides a spinal implant. The spinal implant includes an attachment portion configured to attach to a spinal construct or a bone of a subject, a battery, at least one sensor configured to measure strain, position and/or acceleration of the implant, a transmitter electrically interfaced with an antenna and a processor. The processor is configured to receive, from the at least one sensor, raw strain, position and/or acceleration information. The processor is further configured to execute one or more predefined algorithms, each predefined algorithm configured to process the raw information to determine a health parameter associated with the subject. The processor is further configured to cause the transmitter to transmit at least one determined health parameter to a remote device.

In one aspect, the present disclosure provides a sensing system. The sensing system includes multiple spinal implants. Each spinal implant includes an attachment portion configured to attach to a spinal construct or a bone of a subject, a battery, at least one sensor, a transmitter electrically interfaced with an antenna and a processor. The processor is configured to receive measurement information from the at least one sensor and cause the transmitter to transmit the measurement information to a remote device and/or another implant. Furthermore, at least one implant is configured to receive other measurement information from another implant and cause the transmitter to transmit the other measurement information to the remote device and/or another implant.

### BRIEF DESCRIPTION OF THE DRAWINGS

The accompanying drawings are incorporated into this document and form a part of the specification.

FIG. 1 illustrates an example spinal construct.

FIG. 2 illustrates an example telemetry system.

FIG. 3 provides an exploded view of an example implant.

FIGS. 4-8 illustrate example spinal construct data flow configurations.

FIGS. 9A, 9B, and 10 show flowcharts of a method.

FIG. 11 shows a block diagram of an example of internal hardware that may be used to contain or implement program instructions according to an embodiment.

In the drawings, like reference numbers generally indicate identical or similar elements. Additionally, generally, the left-most digit(s) of a reference number identifies the drawing in which the reference number first appears.

### DETAILED DESCRIPTION

Sensor-enabled spinal implants provide the ability for remote monitoring of patients following surgery to evaluate progression towards recovery. Spinal fusion procedures can involve the use of multiple implants, including pedicle screws, hooks, set screws, rods, cages, rod connectors, tether connectors, cross-links, plates, and interspinous fixation devices. For instance, six pedicle screws are typically placed for a two-level construct, whereas 18 screws could be placed for an eight-level construct, including use of two pedicle screws at each level, dual or quad rods, and multiple cages across different levels for a complex adult procedure.

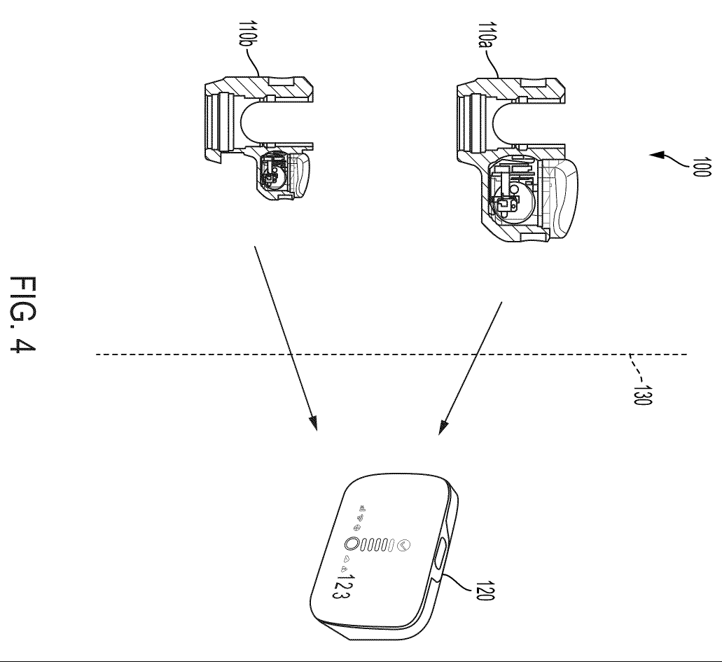

However, communicating with and capturing data from all these devices may be challenging in some applications. For example, any transmitted signal will pass through some amount of tissue and, therefore, be attenuated to some degree. Deeper implants may have to contend with a greater amount of signal attenuation. Furthermore, space-constrained implants may have smaller batteries, or even no battery at all. These implants may be unable to transmit at high power levels, further exacerbating the signal-attenuation issue. Other factors, such as the distance to the external reader 120 (FIG. 4) and varying antenna orientation between implants may cause certain implants to more effectively communicate with the external device 120 than other implants. Furthermore, the antenna orientation may vary between different external devices. Because of the different antenna orientations, different implants may be more or less effective communicating with different external devices. However, to have the most complete understanding of the subject’s condition may require accessing all data from all implants.The systems and methods of this disclosure provide for reliably shepherding data from measurement to reception by the external device.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting.

In some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other and are not necessarily “superior” and “inferior.” Generally, similar spatial references of different aspects or components indicate similar spatial orientation and/or positioning, i.e., that each “first end” is situated on or directed towards the same end of the device.

It is to be appreciated that the Detailed Description section, and not any other section, is intended to be used to interpret the claims. Other sections can set forth one or more but not all exemplary embodiments as contemplated by the inventor(s), and thus, are not intended to limit this disclosure or the appended claims in any way.

The following discussion includes a description of spinal implants 110 as well as telemetry systems that include multiple spinal implants 110 in accordance with the principles of the present disclosure. Reference is made in detail to the exemplary embodiments of the present disclosure, which are illustrated in the accompanying figures.

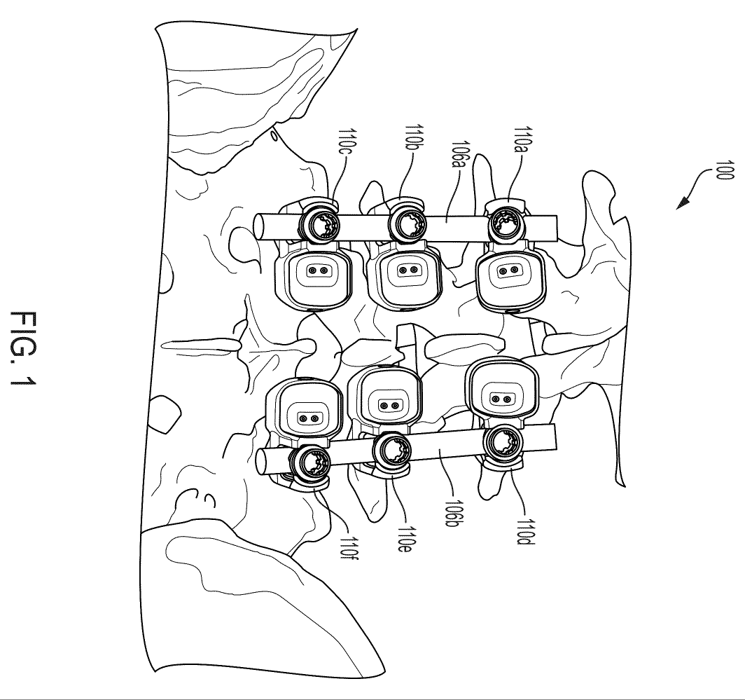

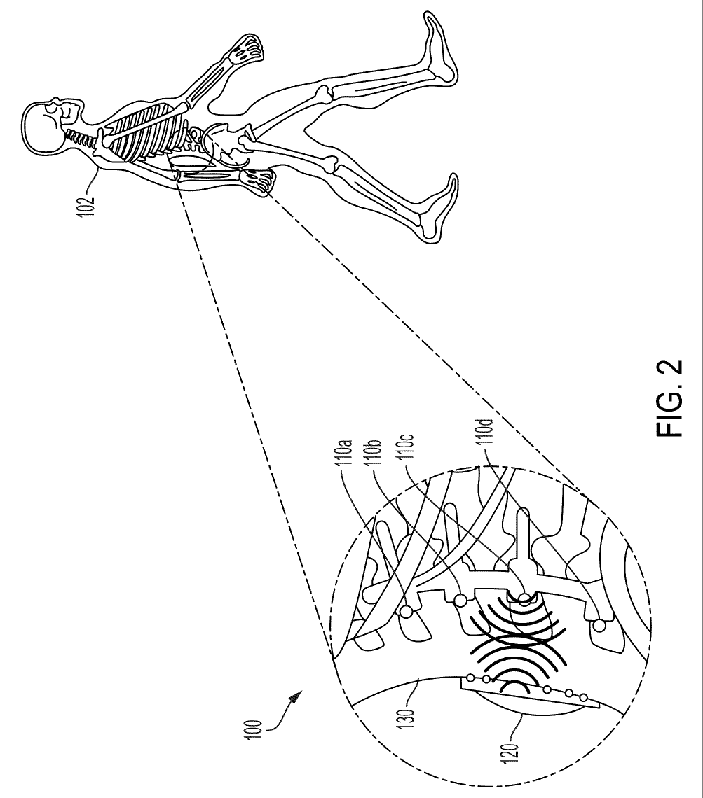

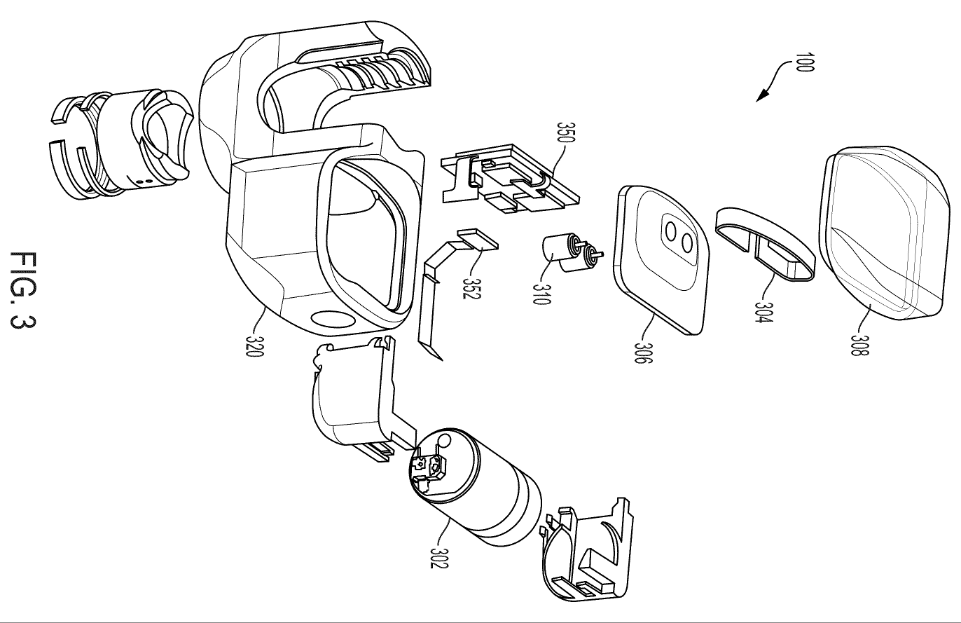

FIG. 1 illustrates an example spinal-fusion construct 100 having multiple separate sensor-equipped implants 110a-f, each of which may have one or more sensors 352 (FIG. 3), such as strain sensors. The sensors 352 may be part of a sensing assembly that may also include additional electronic components. For example, the sensing assembly may include a printed-circuit board on which one or more additional sensors are mounted. In some examples, the sensing assembly may also include signal conditioning electronics, connectors, and other components for interfacing the sensor(s) to other electronic components or mounting the sensor(s) to the implant 110. In some examples, the sensing assembly includes a processor, a transmitter, and/or an antenna 304. Sensors 352 may include, without limitation, force or strain sensors, position sensors, gyroscopes, accelerometers, temperature sensors, and so forth. Some implants 110 may be equipped with an inertial measurement unit (IMU). The IMU may measure the implant’s position, orientation, and/or changes in position and/or orientation, e.g., using a combination of accelerometers, gyroscopes, and/or magnetometers. For example, a six-axis IMU may include a three-axis gyroscope and a three-axis accelerometer. A nine-axis IMU may also include a three-axis magnetometer. To capture daily life human activities, such as walking, or picking up objects, IMUs may have sampling rates of a few hundred Hz. Thus, IMUs may generate a relatively large amount of data, especially compared to sensors that measure more slowly changing parameters, e.g., a temperature sensor. The construct 100 may also include one or more longitudinal members 106a, 106b to add extra support and strength the subject’s spine, and/or prevent movement of vertebrae, e.g., to allow a spinal fusion to heal. In some examples, each implant 110 is secured/anchored to a bone of the subject 102 (FIG. 2). For example, the implant 110 may include a screw configured to anchor the implant 110 to a pedicle of the subject’s vertebra and/or another component of a spinal construct 100, such as longitudinal members 106, a hook member, a cross-link connector, an offset connector, or a hybrid hook-screw member, etc. Implants 110 may have additional uses, including, but not limited to constraining vertebral motion using a tether or ligament tape. Other embodiments within the scope of this disclosure include multiple implant systems, e.g., multiple spinal-fusion constructs 100, and/or individual sensor-equipped implants 110.

During the healing/recovery process, it may be useful to monitor various aspects of the construct 100 or the subject 102. For example, strain sensors 352, and especially differences between strain sensors 352 from different implants 110, may indicate whether the loads are distributed properly among the components of the construct 100. Strain sensors 352 may also indicate failure of the construct 100, e.g., caused by screws pulling out of bones, or implants 110 otherwise becoming detached. Temperature sensors may indicate infection at or near the surgical site. Position and orientation data may reveal information about range of motion, flexibility, or other parameter of the subject’s spine during the healing process. IMU data may also reveal information about the subject’s post-operative activity. For example, individual steps taken by the subject 102 may produce a recognizable “signature” in accelerometer data. Thus, by analyzing sensor data from the accelerometers, the number of steps taken by a subject 102 may be determined.

Referring to FIG. 2, in addition to the one or more sensors, each sensor-equipped implant 110 also includes a transmitter configured to transmit the sensor data to a reader device 120 that is external to the subject 102. That is, the implanted sensors are separated from the external device 120 by a skin boundary 130 of the subject 102. FIG. 2 shows the reader device 120 disposed in proximity to a region of the lower back of a subject 102 and close to the subject’s skin boundary 130. In other examples, the reader device 120 may be disposed elsewhere on the subject 102, or the reader device 120 may be disposed at a location that is remote from the subject 102, such as a bed-side monitor that may be located as much as two meters away. In other examples, the reader device 120 is integrated into a surgical planning system, a robotic navigation system, or other system configured to assist in treatment of the subject 102. Each sensor-equipped implant 110 may also include a power source, such as a battery 302 (FIG. 3) and associated electronics to enable the implant 110 to obtain and transmit the sensor data (e.g., a microprocessor, transceiver, antenna 304, and so forth). In some examples, the battery 302, sensing assembly, and/or associated electronics are housed within a sealed cavity to protect the components. The sealed cavity may be integrated within the implant 110 or may be attached to, e.g., a side of the implant 110.

Referring to FIG. 3, an exploded view of an example implant 110 is shown. In this embodiment, an electronics enclosure 320 is disposed at the side of the implant 110. The electronics enclosure 320 may be a hermetically sealed cavity that houses the sensor 352 (and associated assembly) and other associate electronics components. The electronics associated with the sensing assembly may include a battery 302 or other power source and readout electronics 350. These components may be hermetically sealed within the electronics housing, e.g., by cover 306. As shown, an antenna 304 is located on the side of cover 306 that would face the external reader 120, to allow for more effective transmission of sensor data. The antenna 304 may be enclosed within a header or cap 308 that is configured to protect the antenna 304 and to protect tissue of the patient from damage/irritation from the antenna 304. The readout electronics 350 interface with the antenna 304 via feedthroughs 310 that pass through the cover 306. The example support electronics may include various electronic components in electrical communication with one another. For example, the readout electronics 350 may include a mainboard or other suitable printed circuit board (PCB), which may be electrically connected to an application specific integrated circuit (ASIC), a microcontroller, a transceiver, a charge storage capacitor, and various mechanical electrical sensors (MEMs) such as temperature sensors, position sensors, gyroscopes, and the like. In some embodiments, electronics components may include a non-transitory data store (not illustrated) according to an embodiment, e.g., a memory cell such as a solid-state memory cell or the like. The non-transitory memory data store may store information and/or data from various MEMs sensors 352, for example. A non-transitory data store may be used to store various information. For example, one or more measurements of a strain gauge 352 may be stored in memory. As another example, a unique identifier associated with a load sensing assembly, a component thereof, or the implant 110 may be stored in memory. Additional and/or alternate information or types of information may be stored as is consistent with this disclosure. Additionally, in some embodiments, electronics components may be coated in a material to prevent and/or suppress corrosion, e.g., a conformal coating, an epoxy coating, aerosol coating, or the like.

Certain regions of the subject’s anatomy may impose more strict space constraints for implants than others. For example, the upper thoracic and/or cervical regions of the spine may have inadequate space for receiving a full size (and full featured) implant 110. Instead, there may only be sufficient available volumetric space to receive smaller implants that may be less capable than their full-sized counterparts. These smaller implants 110 may have a reduced-size electronics housing/enclosure, which may necessitate smaller electronic components. For example, these smaller implants 110 may have a reduced-size battery or even no battery at all. To compensate for the smaller battery 302 and associated reduced capacity to store electrical energy, these smaller implants may be specifically designed to reduce battery consumption, so as to achieve a desired battery lifetime. For example, the transmitter may consume significant power while transmitting data. Therefore, the implant 110 may be configured to cause the transmitter to turn off or assume a low-power mode as much as possible (e.g., whenever the implant 110 is not actively transmitting data). The transmitter’s power demand may also be a function of transmitted signal power. That is, transmitting a stronger signal may require more electrical power. Therefore, transmitting a signal strong enough to overcome significant signal attenuation (e.g., for more deeply located implants) may be particularly draining on the battery. In contrast, the power required to operate the processor and/or maintain the memory may be significantly less demanding on battery power. Therefore, these smaller implants may be configured to use data processing to reduce the amount of clinically relevant data to transmit.

Clinical relevance refers to the ability to positively impact a patient's health, function, or survival. It can also refer to information that answers a question that is important to patients or clinicians in determining the patient’s health. Thus, clinically relevant data includes one or more parameters related to the health of the patient, including the integrity and/or proper functioning of a spinal construct 110 or other medical or surgical intervention.

As discussed above, IMUs may generate a relatively large amount of data compared to other sensors, at least because IMUs typically include multiple axes and higher data acquisition rates. Therefore, smaller implants 110 may be configured without an IMU, thus decreasing the amount of data to transmit (and the associated power required to transmit that IMU data). Instead, these smaller implants may have lower data rate sensors, such as strain gauges, temperature sensors, single-axis accelerometers, and the like. Because these sensors produce less data than a typical multi-axis IMU, the overall power required to transmit the data is reduced, allowing for a smaller battery 302 (having reduced capacity) without sacrificing battery lifetime with respect to IMU-equipped implants 110. Under some circumstances, however, eliminating the IMU from the implant 110 may result in the loss of clinically relevant data. That is, under certain circumstances, an IMU may be the best type of sensor to acquire clinically relevant data.

Therefore, in some embodiments, the smaller implants 110 are configured to process the raw IMU data in a way that reduces the amount of data the implant 110 will transmit, while preserving (and transmitting) the clinically relevant information from the IMU data. That is, each smaller implant may be configured only to collect only certain type of sensor data, e.g. strain sensor output, while the larger implant may be configured to collect and transmit more data-intense sensor outputs such as IMU output. This configuration allows the smaller implant to have lower requirement for electrical power, which enables physical size reduction of the implant.

For example, the raw IMU data may include accelerometer data that can be analyzed to determine how many steps the subject 102 took during a period of time. That is, steps taken by the subject 102 may result in a distinctive and recognizable acceleration “signature” that can be measured by accelerometers in one or more axes. Furthermore, a difference in the number of steps sensed by one implant 110 may indicate an issue with that implant or its connection to the construct 100. The implants 110 may be configured to process the accelerometer data to identify the number of step “signatures” in the accelerometer data during a period of time. The implants 110 may then transmit the number of steps to the external reader 120, e.g., for further processing, rather than transmitting the accelerometer data that was processed by the implant 110 to determine the number of steps. Because the data that includes the number of steps is smaller than the raw IMU data, the result is less data transmitted to the external reader 120, while preserving the clinically relevant number of steps sensed by the IMU of the implant 110.

This is only one example of an algorithm for processing raw sensor data to determine a health parameter associated with the subject 102. The implant 110 may be configured to execute multiple distinct algorithms, each algorithm configured to process the raw sensor 352 data to identify, extract, or otherwise determine a different clinically relevant parameter from the raw sensor data. In another example, particular failure modes associated with the construct 100, such as an implant 110 pulling out of a bone, may also result in recognizable “signatures” in IMU data. Therefore, each smaller implant 110 may also be configured to execute algorithms that are configured to determine whether a known construct 100 failure mode or anomaly has occurred. As in the previous example, the implant 110 may transmit an indication of the construct 100 failure mode, rather than transmitting the raw IMU that was processed to determine the construct 100 failure mode. The indication may include an identifier associated with the construct 100 failure mode. For example, the implant 110 may be configured to identify one or more predefined possible states of the spinal construct 100. The predefined possible states may be represented as an enumerated type, such as an integer or label. As in the previous example, because the data that identifies that a construct 100 failure has occurred is smaller than the raw IMU data that was processed to determine that the failure has occurred, the result is less data transmitted to the external reader 120, while preserving the clinically relevant information of the construct 100 failure.

In another example, strain sensor data can be used to determine fusion progression during the patient recovery period. That is, the load borne by the spinal construct may create measurable strain on a spinal rod. As the fusion progresses, the load borne by construct may decrease as the load is gradually taken over by the fusion mass. Strain measurements that fall outside predefined limits may indicate unsatisfactory post-operative progress. Therefore, each smaller implant 110 may also be configured to execute algorithms that are configured to determine whether the strain measured on the spinal rod is within acceptable limits and/or is decreasing at an acceptable rate over time. The algorithm(s) may be applied to data from each individual strain sensor, to data from all strain sensors, and/or to a weighted average of data from particular strain sensors. In some examples, the health parameter is a binary value, e.g., whether the fusion status is adequate or not. In other cases, the heath parameter may be one of a small set of predefined values, such as “good,” “fair,” “poor,” and “bad,” or any other set of values that indicate a relative, qualitative assessment of the state of fusion progress. In some examples, the health parameter may be an objective measurement of average and/or peak load/force/strain applied to the construct, based on one or more strain measurements. As with the previous examples, because the data that indicates fusion status is smaller than the raw strain data that was processed to determine that the status, the result is less data transmitted to the external reader 120, while preserving the clinically relevant information of fusion status.

In some examples, the implant 110 is configured to process IMU data to identify and/or detect a “signature” of an unrecognized off-normal occurrence. That is, the implant 110 may execute an algorithm that is configured to distinguish expected IMU data from unexpected or surprising data. Unexpected data may include measured acceleration within (or outside) particular frequency ranges, greater than defined thresholds, having unexpected cross-correlation with other axes, IMU data that by itself, or in conjunction with other sensor data, indicates inadequate fusion status, and so forth. In an example, an algorithm may use sensor fusion techniques and combine data from multiple sources such as IMU, temperature, and/or strain sensors, etc. to detect anomalies. In another example, an algorithm may use correlated IMU data to detect that the subject has fallen and/or has been involved in an accident. These unexpected and/or off-normal data may indicate that an unusual defect or anomaly related to the construct 100 has occurred or that the post-operative recovery is deviating from expectations in an unexpected way. In these and other examples, the algorithm may be configured to “flag” portions of the IMU data as potentially clinically relevant but requiring further processing. In response to identifying portions of the IMU that may be clinically relevant, the implant 110 may transmit the identified portions of IMU data to the external reader 120. Because these identified portions are a subset of the entire IMU data, the result is less data transmitted to the external reader 120, while preserving the potentially clinically relevant information.

Referring to FIG. 4, an example construct 100 is shown which includes standard size implant 110a and reduced-sized implant 110b. As shown, external device 120 is a base station configured to receive information transmitted by each implant 110a, 110b. In some examples, reduced-sized implant 110b includes a reduced-capacity battery 302 compared to its standard-sized counterpart. To improve battery lifetime, reduced-sized implant 110b may be configured, as described above, to process raw information from one or more of its sensors so as to reduce the amount of data transmitted to the external device 120, while preserving clinically relevant information. Alternatively, reduced-sized implant 110b may be configured to collect a smaller amount of data compared to its standard-sized counterpart. For example, reduced-sized implant 110b may be configured with fewer sensors (e.g., without an IMU) and/or be configured to acquire data at a slower rate.

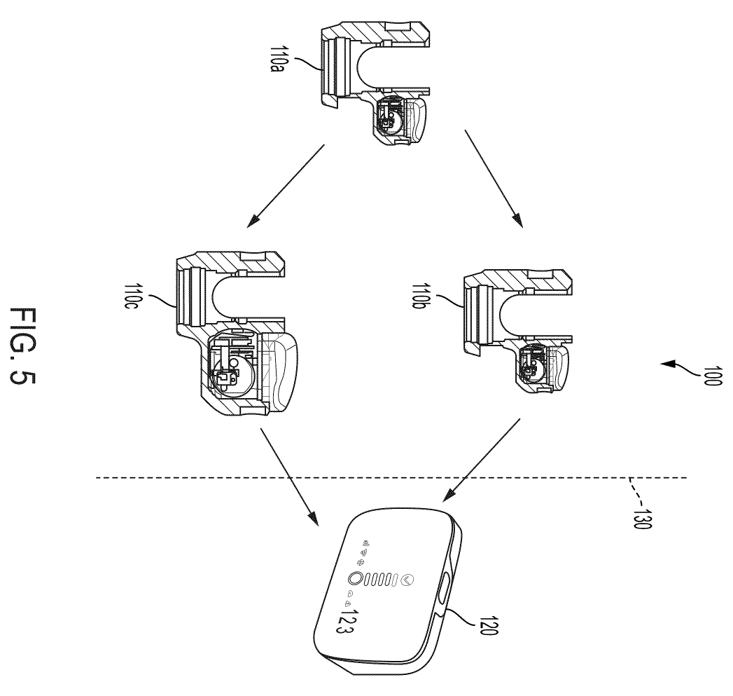

Referring to FIG. 5, another example construct 100 is shown which includes standard size implant 110c and reduced-sized implants 110a and 110b. As shown, implants 110b and 110c are configured to transmit their information to external device 120, in a similar manner to the implants 110 of the example construct 100 shown in FIG. 4. However, implant 110a is configured to transmit its information to implant 110b and/or implant 110c rather than the external reader 120. For example, implant 110a may be implanted more deeply than implants 110b and 110c and/or may include a transmitter that is configured to transmit its signal at a reduced power level than implants 110b and 110c. For these or other reasons, implant 110a may not be able to reliably communicate with the external device 120. To compensate, implant 110b and/or implant 110c may be configured to receive the information transmitted by implant 110a and relay the received information to the external device 120.

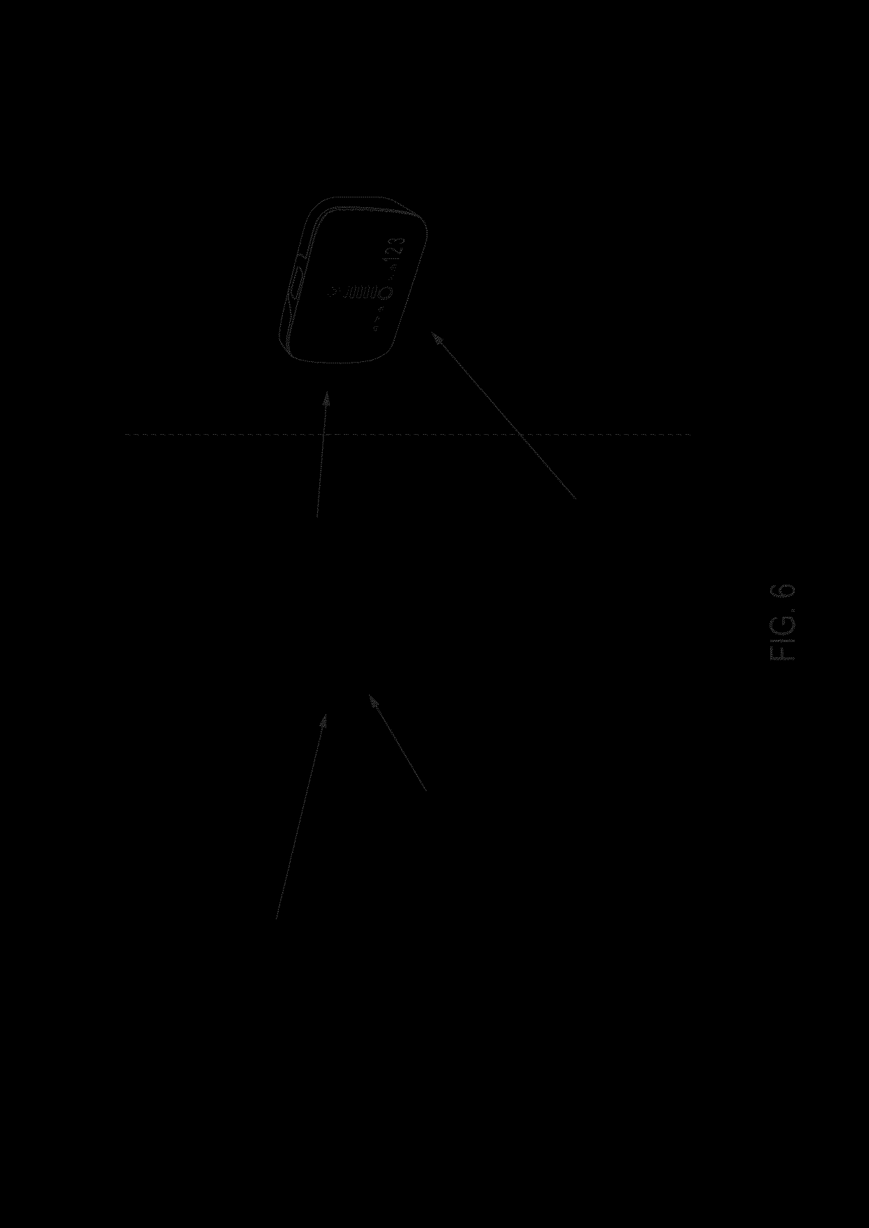

As shown, data from implant 110a can follow two different paths on the way to external device 120. In some examples, data from implant 110a follows all available paths, resulting in redundant data received by the external device 120. In other examples, a single path is selected to avoid redundancy. The path may be selected by implant 110a, e.g., by connecting another implant that responds to a broadcast connection request, such as the first implant 110 that responds to the connection request, or an implant 110 that may not be first, but which has a desired feature, such as high signal strength, or which advertises the ability to perform data-reduction of the raw sensor data. Thus, the system as a whole ensures that data from all implants 110 is reliably transmitted to the reader device 120. In another implementation, 110a may transmit its data to shallower implants in a rotational paradigm so that the receivers can share the data transmission burden throughout the lifetime of the implant. In other examples, the external reader device 120 selects one implant 110b, 110c to communicate with, e.g., based on factors such as received signal strength. Because the reader device 120 selects only one implant 110b, 110c to communicate with, that selection may effectively define the path that data takes from deeply implanted implants 110a (and/or 110d, FIG. 6) to the external reader device.

FIG. 6 shows an example construct 100 that is similar to the example construct 100 shown in FIG. 5, but also including interbody implant 110d. As shown, interbody implant 110d is configured to transmit its sensor information to implant 110b. In some examples, implant 110b is configured to process the information received from implant 110a, e.g., to reduce the amount of data that is transmitted from implant 110b to the external device 120. For example, implant 110b may apply algorithms such as those described above to determine one or more health parameters from the raw sensor data and only transmit the health parameter(s) rather than the raw sensor data. In some examples, implant 110b may include additional data-reducing algorithms. The additional data-reducing algorithms may include processing the received sensor data from implant 110d in combination with other information, such as information from the sensors of implant 110b. For example, implant 110b may compare a health parameter received from implant 110d with a health parameter determined by processing its own sensor data. That is, implant 110b may execute one or more algorithms that are configured to determine whether health-related parameter(s) reported by implant 110d are the same as (or similar to) the health-related parameter(s) determined by processing the raw sensor data of implant 110b. Implant 110b may further reduce the data transmitted to the external device 120 by combining the results of processing the sensor data of implant 110d and implant 110b, e.g., by not sending duplicate results when the health parameter(s) are determined to be the same (or within a threshold difference from each other). The threshold difference for not sending duplicate information may be a simple percentage, such as 10%, or may be based on a clinically relevant difference for the particular health parameter(s).

In some examples, the more deeply implanted implants 110a, 110d, may be configured to use a different (e.g., more energy-efficient) communication standard than the implants 110b, 110c which communicate with the external device 120. For example, implants 110a and 110d may be configured to use the Medical Implant Communication System (MICS) standard to transmit their information to implants 110b, 110c. Implants 110b and 110cmay use Bluetooth or Bluetooth Low Energy (BLE) to transmit information to the external device 120. BLE and MICS operate in different frequency ranges. The higher frequencies of BLE results in greater signal loss through the body and may suffer from interfering signals from other nearby systems such as Wi-Fi. Thus, the more deeply implanted implants 110a, 110d, may be able to transmit their data using MICS at lower power levels than would be required for BLE to go through the same amount of tissue with sufficient remaining signal strength to avoid interference from other sources.

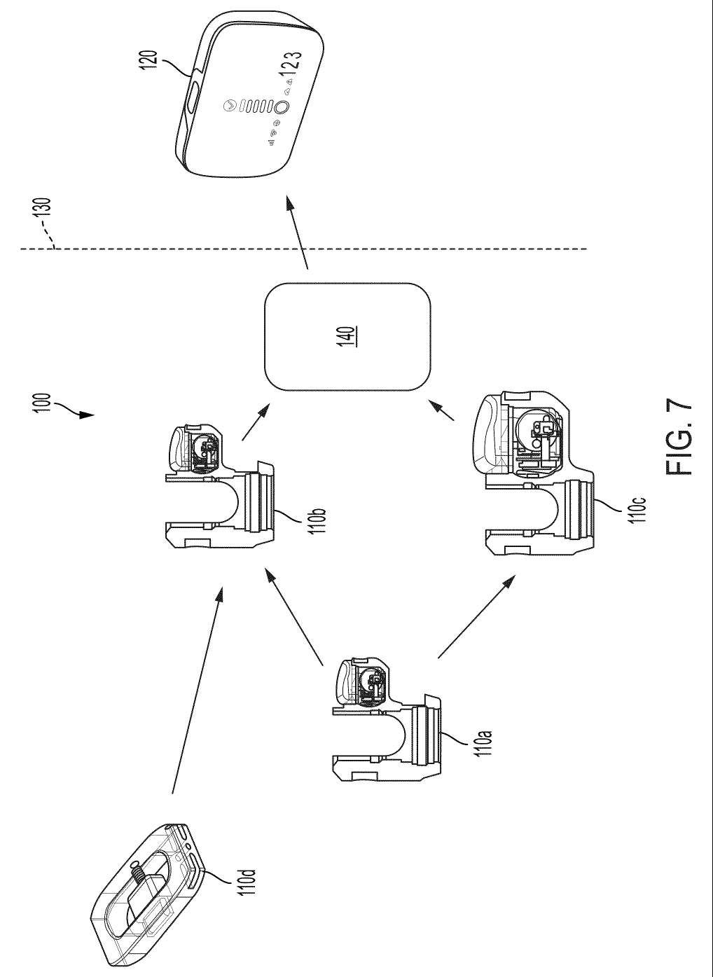

FIG. 7 shows an example construct 100 that is similar to the example construct 100 shown in FIG. 6, with the addition of data courier device 140. Data courier device 140 is a subcutaneous device configured to receive information transmitted from some or all implants 110 of the construct 100 (e.g., at least the deepest implants) and relay the information to the external device 120 (with or without applying one or more data-reducing algorithms). In some examples, data courier device 140 is simply one of the implants 110 that is configured to act as the data courier/data concentrator. In other examples, data courier device 140 does not function as an implant (i.e., does not serve in a role of supporting or stabilizing the subject’s spine). Instead, data courier device 140 may have a form factor that is different than an implant 110. In some examples, the data courier device 140 is configured to attach to the spinal construct 100, e.g., to have a fixed location with respect to the implants 110. In other examples, the data courier device 140 may be close to the skin 130 of the subject 102, e.g., to reduce the amount of tissue that the signal must pass through to get to the external reader device 120 and, thus, the attenuation of the signal passing through the tissue. In some examples, the external device 120 only communicates with the data courier device 140 and does not communicate directly with any other implants 110. That is, all the data from the implants passes through the data courier device 140 before being transmitted to the external device 120. Alternatively, some or all implants 110 communicate directly with the external reader 120. As in the previous examples, one or more implants may execute one or more algorithms to reduce the raw sensor data to clinically relevant health parameters associated with subject 102 and/or the construct 100.

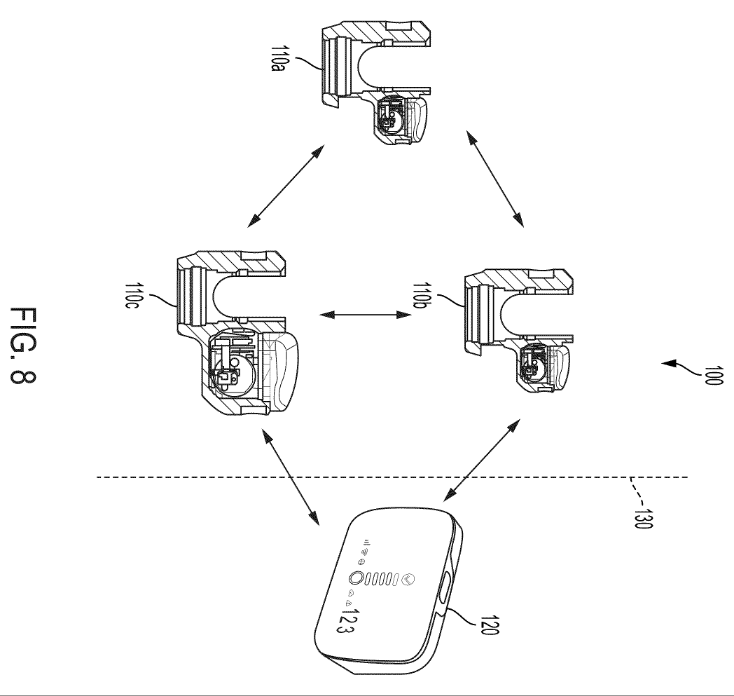

FIG. 8 shows an example construct 100 in which the implants 110 are configured to receive and store information transmitted by other implants. That is, the implants 110 may share their data with each other. As shown, each implant 110 transmits its data to all other implants. Thus, all data is fully replicated at each implant. The external reader device 120 is then able to download the data by communicating with a single implant 110. In other embodiments, the construct 110 includes subsets of implants 110, where all data is fully replicated within each subset. Thus, the external reader device 120 is then able to download the data by communicating with any single implant 110 from each subset. The data redundancy can be achieved in a number of different ways. For example, each implant 110 may communicate directly with each other implant (e.g., in its subset). Alternatively, each implant may broadcast its data to any and all other implants 110. In another embodiment, one implant 110 may transmit its data to a second implant 110 which may aggregate the data with its own data before transmitting the combined data to a third implant 110 and so on. Furthermore, as described above, data-reduction processing may be performed by any of the implants 110 along the way. An advantage of full data replication within the implants 110 (or a subset of the implants 110) is that any one of the implants is able to perform global processing, e.g., comparing salient features of data obtained at each of the implants 110.

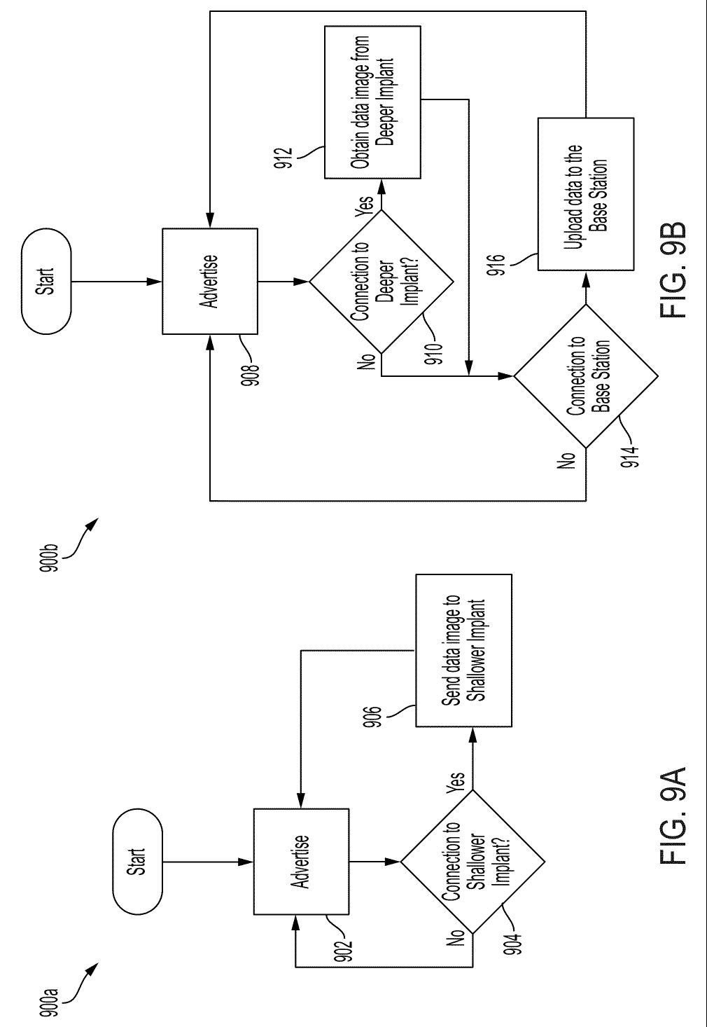

FIG. 9A shows a flowchart 900a of a method of communicating between implants 110. The method is suitable for implants 110 that will not communicate with the external reader 120, such as deeply implanted and/or low-power implants 110. At step 902, the method includes advertising for a network connection, e.g., with another implant 110. In some examples, the implant 110 only advertises for a network connection when it has data to transmit, e.g., to conserve power. Alternatively, the implant 110 may advertise for a network connection, e.g., periodically, whenever it is powered on. Step 902 may include broadcasting a request to connect with another implant 110, e.g., using a standard communication protocol. Step 902 may also include receiving a reply from another implant 110 and connecting with the other implant 110, if appropriate. For example, it may only be appropriate to connect with the other implant 110 if the other implant 110 has a desired feature. Desired features may include the ability to communicate with an external reader device 120, the ability to perform data reduction, or having sufficient signal strength for reliable communication (e.g., as detected by the implant 110), among others. If the desired criteria are met, the implant 110 may connect with the other implant 110. At step 904, the method includes determining whether the implant 110 has established a connection to another implant 110 (e.g., a shallower implant 110). If not, the method reverts to step 902. If, however, the implant 110 does have a connection with another implant 110, the method continues to step 906. At step 906, the method includes sending information to the other implants 110 over the connection. For example, the implant may transmit its sensor data to the other implant 110. Alternatively, the implant 110 may transmit a health parameter that was determined from the sensor data. After sending information to the other implant, the method continues at step 902.

FIGS. 9B shows a flowchart 900b of a method of communicating between implants 110. This method is suitable for implants 110 that will communicate with the external reader 120. At step 908, the method includes advertising for a network connection, e.g., with another implant 110 and/or the external device 120 (e.g., base station). That is, step 908 may be substantially similar to step 902 described above, while also including advertising to the external device 120. Also similar to step 902 described above, step 908 may also include establishing connections with other implants 110 and/or the external device 120. At step 910, the method includes determining whether the implant 110 has an existing connection to another implant 110 (e.g., a deeper implant 110). If so, the method includes, at step 912, receiving information from the other implant 110. As described above the information may be raw sensor data or may be a health parameter that was determined from the raw sensor data by the other implant 110. The method also includes determining, at step 914, whether the implant has an existing connection to an external reader device 120. If so, the method includes, at step 916, transmitting its information (e.g., either raw or processed) and/or information received from other implants 110 (e.g., either as received or after further processing) to the external device 120.

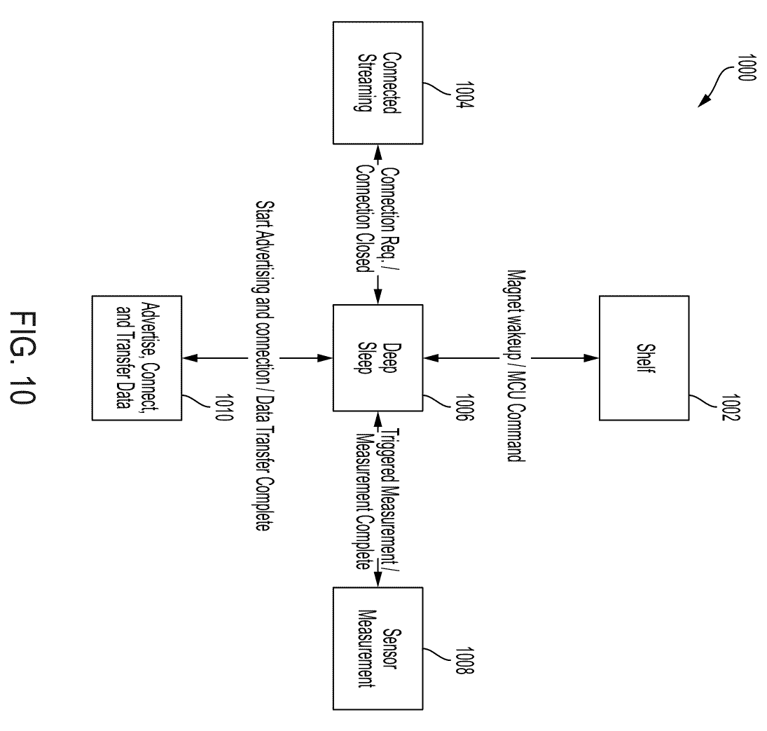

FIG. 10 shows a state diagram 1000 showing an example implant lifecycle. State 1002 represents a non-operational state of the implant 110. The implant may be in state 1002 while it is in storage (e.g., in packaging and stored on a shelf) and not yet configured for operation. In some examples, the implant 110 will remain in the non-operational state 1002 until it receives a command, signal, or other indication to enter an operational state (e.g., 1004, 1006, 1008, 1010). A surgeon (or other medical professional) may configure the implant 110 for use before or during a medical procedure, e.g., at or near the time the implant 110 is installed. For example, the implant 110 may be configured to respond to a magnetic field applied by the medical professional. In other examples, the implant 110 may receive a signal from a button, a transmitter coil or a command from wireless transmission. After receiving the signal, the implant 110 may enter a quiescent state 1006. In this “deep sleep” state, the implant may wait for a signal or other indication to perform specific operations. For example, the implant 110 may receive a trigger to perform data acquisition. The trigger may be generated by a timer or other circuit that indicates that the implant 110 should perform the data acquisition. For example, a timer may cause the implant 110 to take a temperature measurement at several different times throughout a day, such as 5 times per day. In another example, a lower-power-consumption sensor, such as an accelerometer may detect an acceleration above a threshold and, in response, signal the implant 110 to enable and obtain measurements from one or more higher-power-consumption sensors, such as an IMU. In response, the implant 110 may enter a measurement state 1008. In this state, the implant 110 may obtain measurements from one or more sensors 352 and one or more sensors on the readout electronics 350 . Furthermore, the implant 110 may perform data reduction (e.g., including determining one or more health parameters, as described above). The implant 110 may store the sensor data and/or health parameters in memory or otherwise preserve the information for subsequent transmission. After storing the data, the implant 110 may return to the quiescent state 1006. In some examples, the implant 110 may enter a data transfer state 1010. In this state 1010, the implant performs steps of flowcharts 900a and/or 900b. That is, the implant may advertise for connections, connect with other implants 110, and/or transmit or receive information from the other implant 110. The implant may enter data transfer state 1010 in response to a trigger from a timer (e.g., periodically, such as daily at midnight) and/or in response to receiving a trigger from a sensor such as an accelerometer. After data transfer activities are complete, the implant 110 may return to the quiescent state 1006. Similarly, the implant 110 may enter a data streaming state 1004. In this state 1004, the implant may continuously measure and stream the data to an external reader 120, for example during a clinic visit or in the operating room. After streaming data, the implant 110 may revert to the quiescent state 1006.

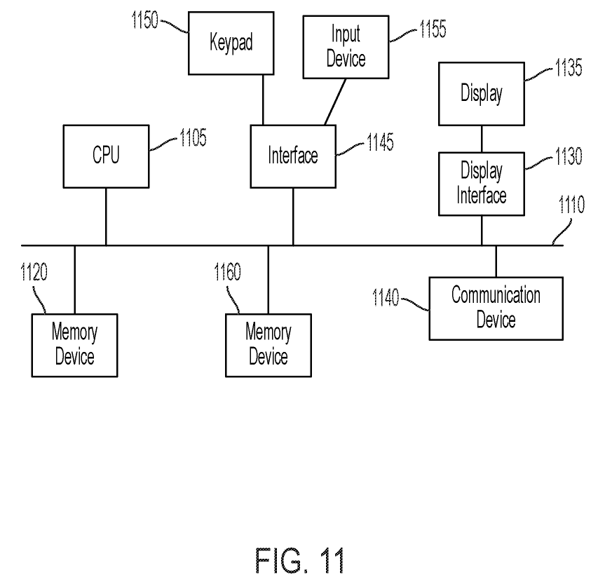

FIG. 11 illustrates example hardware that may be used to contain or implement program instructions. A bus 1110 serves as the main information highway interconnecting the other illustrated components of the hardware. Central Processing Unit (CPU) 1105 is the central processing unit of the system, performing calculations and logic operations required to execute a program. CPU 1105, alone or in conjunction with one or more of the other elements disclosed in FIG. 11, is an example of a processor as such term is used within this disclosure. Read only memory (ROM) and random-access memory (RAM) constitute examples of non-transitory computer-readable storage media 1120, memory devices or data stores as such terms are used within this disclosure.

Program instructions, software or interactive modules for providing the interface and performing any querying or analysis associated with one or more data sets may be stored in the memory device 1160. Optionally, the program instructions may be stored on a tangible, non-transitory computer-readable medium such as a compact disk, a digital disk, flash memory, a memory card, a universal serial bus (USB) drive, an optical disc storage medium and/or other recording medium.

An optional display interface 1130 may permit information from the bus 1110 to be displayed on the display 1135 in audio, visual, graphic or alphanumeric format. Communication with external devices may occur using various communication devices and/or ports 1140. A communication port 1140 may be attached to a communications network, such as the Internet or an intranet. Communication devices may include wireless transceivers for receiving and/or relaying telemetry. That is, a transceiver may comprise both a transmitter and a receiver.

The hardware may also include an interface 1145 which allows for receipt of data from input devices such as a keypad 1150 or other input device 1155 such as a touch screen, a remote control, a pointing device, a video input device and/or an audio input device.

While this disclosure describes example embodiments for example fields and applications, it should be understood that the disclosure is not limited to the disclosed examples. Other embodiments and modifications thereto are possible and are within the scope and spirit of this disclosure. For example, and without limiting the generality of this paragraph, embodiments are not limited to the software, hardware, firmware, and/or entities illustrated in the figures and/or described in this document. Furthermore, embodiments (whether or not explicitly described) have significant utility to fields and applications beyond the examples described in this document.

Embodiments have been described in this document with the aid of functional building blocks illustrating the implementation of specified functions and relationships. The boundaries of these functional building blocks have been arbitrarily defined in this document for the convenience of the description. Alternate boundaries can be defined as long as the specified functions and relationships (or their equivalents) are appropriately performed. Also, alternative embodiments can perform functional blocks, steps, operations, methods, etc. using orderings different than those described in this document.

The features from different embodiments disclosed herein may be freely combined. For example, one or more features from a method embodiment may be combined with any of the system or product embodiments. Similarly, features from a system or product embodiment may be combined with any of the method embodiments herein disclosed.

References in this document to “one embodiment,” “an embodiment,” “an example embodiment,” or similar phrases, indicate that the embodiment described can include a particular feature, structure, or characteristic, but every embodiment can not necessarily include the particular feature, structure, or characteristic. Moreover, such phrases are not necessarily referring to the same embodiment. Furthermore, when a particular feature, structure, or characteristic is described in connection with an embodiment, it would be within the knowledge of persons skilled in the relevant art(s) to incorporate such feature, structure, or characteristic into other embodiments, whether or not explicitly mentioned or described in this document. Additionally, some embodiments can be described using the expression “coupled” and “connected” along with their derivatives. These terms are not necessarily intended as synonyms for each other. For example, some embodiments can be described using the terms “connected” and/or “coupled” to indicate that two or more elements are in direct physical or electrical contact with each other. The term “coupled,” however, can also mean that two or more elements are not in direct contact with each other, but still co-operate or interact with each other.

In this document, “electronic communication” refers to the transmission of data via one or more signals between two or more electronic devices, whether through a wired or wireless network, and whether directly or indirectly via one or more intermediary devices. Devices are “communicatively connected” if the devices are able to send and/or receive data via electronic communication. Any communication unit may include a transmitter and receiver. Telemetry refers to electronic communication of sensor data. Wireless telemetry may use BLUETOOTH LOW ENERGY (BLE) protocols, Zigbee, Wimax, WiFi, near field communications (NFC), tissue conductance communication (TCC), Medical Implant Communication System (MICS), RFID, or other wireless communication protocols. TCC is an intrabody communication protocol which allows implantable devices to communicate with each other.

The invention may be further described by reference to the following numbered clauses:

Clause 1. A spinal implant comprising:

an attachment portion configured to attach to a spinal construct or a bone of a subject;

a battery;

at least one sensor configured to measure strain, temperature, position and/or acceleration of the implant;

a transmitter electrically interfaced with an antenna; and

a processor configured to:

receive, from the at least one sensor, raw strain, temperature, position and/or acceleration information;

execute one or more predefined algorithms, each predefined algorithm configured to process the raw information to determine a health parameter associated with the subject; and

cause the transmitter to transmit at least one determined health parameter to a remote device.

Clause 2. The spinal implant of clause 1, wherein the processor is configured to cause the transmitter to transmit the raw information that was received from at least one sensor.

Clause 3. The spinal implant of clause 1, further comprising a sealed cavity for supporting the battery and processor within.

Clause 4. The spinal implant of clause 1, wherein the processor is further configured to cause the transmitter to be in a low-power mode when not transmitting.

Clause 5. The spinal implant of clause 1, wherein the heath parameter comprises a state of the spinal construct, the state of the spinal construct comprising one of a set of predefined states.

Clause 6. The spinal implant of clause 1, wherein the health parameter associated with the subject indicates a fusion status.

Clause 7. The spinal implant of clause 1, wherein the processor is further configured to:

cause the spinal implant to receive raw sensor information from another implant;

execute the one or more predefined algorithms to process the received raw sensor information to determine a second health parameter associated with the subject; and

cause the transmitter to transmit, using the antenna, the second health parameter to the remote device.

Clause 8. The spinal implant of clause 7, wherein the processor is further configured to cause the spinal implant to:

receive the raw sensor information from the other implant using a first wireless transmission standard; and

cause the transmitter to transmit the second health parameter to the remote device using a second wireless transmission standard, the second wireless transmission standard different than the first wireless transmission standard.

Clause 9. The spinal implant of clause 1, wherein the processor is further configured to:

cause the spinal implant to receive raw sensor information from another implant; and

cause the transmitter to transmit, using the antenna, the received raw sensor information to the remote device.

Clause 10. The spinal implant of clause 1, wherein the attachment portion comprises a pedicle screw.

Clause 11. The spinal implant of clause 1, wherein:

the health parameter associated with the subject is a number of steps taken by the subject; and

at least one predefined algorithm is configured to process the raw information to determine the number of steps taken by the subject.

Clause 12. A sensing system comprising:

a plurality of spinal implants, each spinal implant comprising:

an attachment portion configured to attach to a spinal construct or a bone of a subject;

a battery;

at least one sensor;

a transmitter electrically interfaced with an antenna; and

a processor configured to:

receive measurement information from the at least one sensor; and

cause the transmitter to transmit the measurement information to a remote device and/or another implant;

wherein at least one implant of the plurality of spinal implants is configured to:

receive other measurement information from another implant; and

cause the transmitter to transmit the other measurement information to the remote device and/or another implant.

Clause 13. The sensing system of clause 12, wherein each spinal implant is configured to transmit its measurement information to every other spinal implant.

Clause 14. The sensing system of clause 12, wherein at least one spinal implant is configured to cause the transmitter to transmit the measurement information to another implant without transmitting its measurement information to the remote device.

Clause 15. The sensing system of clause 12, further comprising:

a subcutaneous data courier device configured to:

receive measurement information from at least one other spinal implant; and

relay the received measurement information to the remote device;

wherein the subcutaneous data courier device is not a spinal implant.

Clause 16. The sensing system of clause 12, further comprising at least one data-reducing implant comprising:

at least one sensor;

a transmitter electrically interfaced with an antenna; and

a processor configured to:

receive measurement information from the at least one sensor;

execute one or more predefined algorithms, each predefined algorithm configured to process the measurement information to determine a health parameter associated with the subject; and

cause the transmitter to transmit at least one determined health parameter to the remote device.

Clause 17. The sensing system of clause 12, wherein at least one spinal implant is configured to cause the transmitter to transmit the measurement information to the remote device.

Clause 18. A method of monitoring a subject, the method comprising:

using the sensing system of clause 1, receiving, by the remote device, the at least one determined health parameter.

Clause 18. A method of monitoring a subject, the method comprising:

using the sensing system of clause 1, receiving, by the remote device, the at least one determined health parameter.

Clause 19. The method of monitoring the subject of clause 18, wherein:

the health parameter associated with the subject is an indication of infection; and

at least one predefined algorithm is configured to compare temperature sensor information to a threshold value to determine the indication of infection.

Clause 20. The method of monitoring the subject of clause 18, wherein:

the health parameter associated with the subject is an indication of a fusion status; and

at least one predefined algorithm is configured to process strain sensor information to determine the indication of the fusion status.

Clause 21. The spinal implant of clause 1, wherein the processor is configured to cause the transmitter to transmit the at least one determined health parameter to the remote device without transmitting the raw information that was processed to determine the health parameter.

Clause 22. The spinal implant of clause 1, further comprising at least one additional sensor, wherein the processor is further configured to:

receive measurement information from the at least one additional sensor; and

cause the transmitter to transmit the measurement information to the remote device.

Clause 23. The spinal implant of clause 22, wherein the at least one additional sensor is configured to measure temperature or strain.

Clause 24. A method of monitoring a subject, the method comprising:

using the sensing system of clause 16, receiving, by the remote device, the at least one determined health parameter.

The breadth and scope of this disclosure should not be limited by any of the above-described example embodiments but should be defined only in accordance with the following claims and their equivalents.

What is claimed is:

1. A spinal implant comprising:
an attachment portion configured to attach to a spinal construct or a bone of a subject;
a battery;
at least one sensor configured to measure strain, temperature, position and/or acceleration of the implant;
a transmitter electrically interfaced with an antenna; and
a processor configured to:
receive, from the at least one sensor, raw strain, temperature, position and/or acceleration information;
execute one or more predefined algorithms, each predefined algorithm configured to process the raw information to determine a health parameter associated with the subject; and
cause the transmitter to transmit at least one determined health parameter to a remote device.

2. The spinal implant of claim 1, wherein the processor is configured to cause the transmitter to transmit the raw information that was received from the at least one sensor.

3. The spinal implant of claim 1, further comprising a sealed cavity for supporting the battery and processor within.

4. The spinal implant of claim 1, wherein the processor is further configured to cause the transmitter to be in a low-power mode when not transmitting.

5. The spinal implant of claim 1, wherein the heath parameter comprises a state of the spinal construct, the state of the spinal construct comprising one of a set of predefined states.

6. The spinal implant of claim 1, wherein the health parameter associated with the subject indicates a fusion status.

7. The spinal implant of claim 1, wherein the processor is further configured to:
cause the spinal implant to receive raw sensor information from another implant;
execute the one or more predefined algorithms to process the received raw sensor information to determine a second health parameter associated with the subject; and
cause the transmitter to transmit, using the antenna, the second health parameter to the remote device.

8. The spinal implant of claim 7, wherein the processor is further configured to cause the spinal implant to:
receive the raw sensor information from the other implant using a first wireless transmission standard; and
cause the transmitter to transmit the second health parameter to the remote device using a second wireless transmission standard, the second wireless transmission standard different than the first wireless transmission standard.

9. The spinal implant of claim 1, wherein the processor is further configured to:
cause the spinal implant to receive raw sensor information from another implant; and
cause the transmitter to transmit, using the antenna, the received raw sensor information to the remote device.

10. The spinal implant of claim 1, wherein the attachment portion comprises a pedicle screw.

11. The spinal implant of claim 1, wherein:
the health parameter associated with the subject is a number of steps taken by the subject; and
at least one predefined algorithm is configured to process the raw information to determine the number of steps taken by the subject.

12. A sensing system comprising:
a plurality of spinal implants, each spinal implant comprising:
an attachment portion configured to attach to a spinal construct or a bone of a subject;
a battery;
at least one sensor;
a transmitter electrically interfaced with an antenna; and
a processor configured to:
receive measurement information from at least one sensor; and
cause the transmitter to transmit the measurement information to a remote device and/or another implant;
wherein at least one implant of the plurality of spinal implants is configured to:
receive other measurement information from another implant; and
cause the transmitter to transmit the other measurement information to the remote device and/or another implant.

13. The sensing system of claim 12, wherein each spinal implant is configured to transmit its measurement information to every other spinal implant.

14. The sensing system of claim 12, wherein at least one spinal implant is configured to cause the transmitter to transmit the measurement information to another implant without transmitting its measurement information to the remote device.

15. The sensing system of claim 12, further comprising:
a subcutaneous data courier device configured to:
receive measurement information from at least one other spinal implant; and
relay the received measurement information to the remote device;
wherein the subcutaneous data courier device is not a spinal implant.

16. The sensing system of claim 12, further comprising at least one data-reducing implant comprising:
at least one sensor;
a transmitter electrically interfaced with an antenna; and
a processor configured to:
receive measurement information from the at least one sensor;
execute one or more predefined algorithms, each predefined algorithm configured to process the measurement information to determine a health parameter associated with the subject; and
cause the transmitter to transmit at least one determined health parameter to the remote device.

17. The sensing system of claim 12, wherein at least one spinal implant is configured to cause the transmitter to transmit the measurement information to the remote device.

18. A method of monitoring a subject, the method comprising:
using the sensing system of claim 1, receiving, by the remote device, at least one determined health parameter.

19. The method of monitoring the subject of claim 18, wherein:
the health parameter associated with the subject is an indication of infection; and
at least one predefined algorithm is configured to compare temperature sensor information to a threshold value to determine the indication of infection.

20. The method of monitoring the subject of claim 18, wherein:
the health parameter associated with the subject is an indication of a fusion status; and
at least one predefined algorithm is configured to process strain sensor information to determine the indication of the fusion status.
